SEQUENCE LISTING (34 sequences)

SEQ ID NO: 1 - protein, 5 aa, synthetic construct
EFSMH

SEQ ID NO: 2 - protein, 17 aa, Synthetic construct
GINPNHGGSSYNQKFKG

SEQ ID NO: 3 - protein, 14 aa, Synthetic construct
RGDYYGNLYYAMDY

SEQ ID NO: 4 - protein, 5 aa, Synthetic construct
AYALH

SEQ ID NO: 5 - protein, 17 aa, Synthetic construct
GINPNHGGVSYNQKFKD

SEQ ID NO: 6 - protein, 11 aa, Synthetic construct
FRWDLYYGMDY

SEQ ID NO: 7 - protein, 5 aa, Synthetic construct
EYAMH

SEQ ID NO: 8 - protein, 17 aa, Synthetic construct
GINPNHGGTSNNQKFKG

SEQ ID NO: 9 - protein, 9 aa, Synthetic construct
WGTDPFFAY

SEQ ID NO: 10 - protein, 11 aa, Synthetic construct
KASQSVSNDVV

SEQ ID NO: 11 - protein, 7 aa, Synthetic construct
YASNRYT

SEQ ID NO: 12 - protein, 9 aa, Synthetic construct
QQDYSSPWA

SEQ ID NO: 13 - protein, 10 aa, Synthetic construct
SDNSNMIYMY

SEQ ID NO: 14 - protein, 7 aa, Synthetic construct
DTSRLAS

SEQ ID NO: 15 - protein, 9 aa, Synthetic construct
HQRSIYSWT

SEQ ID NO: 16 - protein, 11 aa, Synthetic construct
KASQDVSTAVA

SEQ ID NO: 17 - protein, 7 aa, Synthetic construct
WASTRHT

SEQ ID NO: 18 - protein, 9 aa, Synthetic construct
HQHYAPPWT

SEQ ID NO: 19 - protein, 142 aa, synthetic construct
MGWSWIFLFLLSGTAGVLSEVQLQQSGPELVKPGASVKISCKTSGYTFTEFSMHWVQQSHGKSLEWIGGINPNHGGSSYNQKFKGKATLTVDKSSSTAYMELRSLTSEDSAVYYCARRGDYYGNLYYAMDYWGQGTSVTVSS

SEQ ID NO: 20 - protein, 139 aa, synthetic construct
MGWSWIFLFLLSGTAGVLSEVQLQQSGPELVRPGASVKKSCKTSGYTFTAYALHWVKQSHGTSLEWIGGINPNHGGVSYNQKFKDKATLTVDKSSSTAYMELRSLTSEDSAVYYCARFRWDLYYGMDYWGQGTSVIVAS

SEQ ID NO: 21 - protein, 137 aa, synthetic construct
MGWSWIFLFLLSGTAGVLSEVQLQQSGPELVKPGASVKISCKTSGYTFTEYAMHWVKQSHGKSLEWIGGINPNHGGTSNNQKFKGKATLTVDKSSSTAYMDFRSLTSEDSAVYYCARWGTDPFFAYWGQGTLVTVSA

SEQ ID NO: 22 - protein, 129 aa, synthetic construct
MKSQTQVFVFLLLCVSGAHGSIVMTQTPKFLLVSVGDSITITCKASQSVSNDVVWYQQKPGQSPKLLIYYASNRYTGVPDRFTGSGYGTDFTFTITTVQAEDLAVYFCQQDYSSPWAFGGGTKLEIKRA

SEQ ID NO: 23 - protein, 130 aa, synthetic construct
MDFQVQIFSFLLISASVILSRGQIVLTQSPAIISVSSGEKVTMPCSDNSNMIYMYWNLWKPGTSPKRWIYDTSRLASGVPARFSGSGFGTSFSLTVSSMEAEDAATYYCHQRSIYSWTFGGGTKLEIKRA

SEQ ID NO: 24 - protein, 129 aa, synthetic construct
MESQIQAFVFVFLWLSGVDGDIVMTQSHKFMSTSVGDRVSITCKASQDVSTAVAWYQQKPGQSPKLLIYWASTRHTGVPDRFTGSGSGTDYTLTITSVQAEDLTLYYCHQHYAPPWTFGGGTKLEIKRA

SEQ ID NO: 25 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 26 - protein, 324 aa, synthetic construct; identical to SEQ ID NO: 25

SEQ ID NO: 27 - protein, 324 aa, synthetic construct; identical to SEQ ID NO: 25

SEQ ID NO: 28 - protein, 466 aa, synthetic construct
MGWSWIFLFLLSGTAGVLSEVQLQQSGPELVKPGASVKISCKTSGYTFTEFSMHWVQQSHGKSLEWIGGINPNHGGSSYNQKFKGKATLTVDKSSSTAYMELRSLTSEDSAVYYCARRGDYYGNLYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 29 - protein, 463 aa, synthetic construct
MGWSWIFLFLLSGTAGVLSEVQLQQSGPELVRPGASVKKSCKTSGYTFTAYALHWVKQSHGTSLEWIGGINPNHGGVSYNQKFKDKATLTVDKSSSTAYMELRSLTSEDSAVYYCARFRWDLYYGMDYWGQGTSVIVASAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 30 - protein, 461 aa, synthetic construct
MGWSWIFLFLLSGTAGVLSEVQLQQSGPELVKPGASVKISCKTSGYTFTEYAMHWVKQSHGKSLEWIGGINPNHGGTSNNQKFKGKATLTVDKSSSTAYMDFRSLTSEDSAVYYCARWGTDPFFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 31 - protein, 234 aa, synthetic construct
MKSQTQVFVFLLLCVSGAHGSIVMTQTPKFLLVSVGDSITITCKASQSVSNDVVWYQQKPGQSPKLLIYYASNRYTGVPDRFTGSGYGTDFTFTITTVQAEDLAVYFCQQDYSSPWAFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 235 aa, synthetic construct
MDFQVQIFSFLLISASVILSRGQIVLTQSPAIISVSSGEKVTMPCSDNSNMIYMYWNLWKPGTSPKRWIYDTSRLASGVPARFSGSGFGTSFSLTVSSMEAEDAATYYCHQRSIYSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 33 - protein, 234 aa, synthetic construct
MESQIQAFVFVFLWLSGVDGDIVMTQSHKFMSTSVGDRVSITCKASQDVSTAVAWYQQKPGQSPKLLIYWASTRHTGVPDRFTGSGSGTDYTLTITSVQAEDLTLYYCHQHYAPPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 34 - protein, 333 aa, synthetic construct
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0098866 A1
(43) Pub. Date: Apr. 9, 2026

(54) TACI ANTIBODY AND USE THEREOF

(71) Applicant: REMEGEN CO., LTD., Yantai (CN)

(72) Inventors: Xuejing YAO, Yantai (CN); Shasha YUN, Yantai (CN); Baopeng ZHANG, Yantai (CN); Dongxue ZHU, Yantai (CN); Yuan HU, Yantai (CN); Ling XING, Yantai (CN)

(21) Appl. No.: 19/110,816

(22) PCT Filed: Sep. 14, 2023
(86) PCT No.: PCT/CN2023/118680

§ 371 Date: Mar. 12, 2025

(30) Foreign Application Priority Data
Sep. 14, 2022 (CN) 202211113801.X

(51) Int. Cl. G01N 33/68; C07K 16/28

(52) U.S. Cl. CPC G01N 33/6863; C07K 16/2878; C07K 2317/92; G01N 2333/70578

(57) ABSTRACT

Provided are a human B lymphocyte stimulator receptor (TACI) antibody having high specificity and high affinity for TACI, and a method for detecting TACI or a TACI-Fc fusion protein by using the TACI antibody. By means of the method, the content of a free TACI or TACI-Fc fusion protein drug in a patient can be detected, and the level of drug absorption in the patient is effectively known according to the content of the drug in the patient, thereby achieving the effects of evaluating the illness state, monitoring the efficacy, and determining the prognosis of a patient with an autoimmune disease.

### FIELD

The present disclosure relates to the field of biomedicine, and in particular to an antibody (TACI) as a B lymphocyte stimulator receptor and use thereof.

### BACKGROUND

Autoimmune diseases (AIDs) are conditions in which the immune system attacks the body's own tissues due to loss of tolerance to self-antigens, ultimately leading to damage in tissues and organs in various systems. Recent large-scale epidemiological studies indicate that the global incidence of autoimmune diseases has reached 5% and 8%, resulting in a huge clinical demand. According to the American Autoimmune Related Diseases Association (AARDA), there are currently more than 100 autoimmune diseases. Prevalent autoimmune diseases include systemic lupus erythematosus (SLE), rheumatoid arthritis (RA), psoriatic arthritis (PsA), myasthenia gravis (MG), multiple sclerosis (MS) and sicca syndrome (SS).

Overexpression of BLyS (B lymphocyte stimulator) and APRIL (a proliferation-inducing ligand) is one of causes of a number of autoimmune diseases. Clinical studies have demonstrated that the concentration of BLyS is often positively correlated with the severity of autoimmune diseases. Consequently, blocking the actions of BLyS and APRIL has been considered as an effective mean for treating autoimmune diseases. BLyS is a new member of the TNF superfamily identified in 1999. BLyS exerts the functions of regulating the maturation of B lymphocytes, promoting the conversion of immunoglobulin classes, assisting T cell activation and participating in autoimmunity by binding to its corresponding receptors. There are currently three known BLyS receptors: BCMA (B cell maturation antigen), BAFF-R (BAFF receptor, BR3) and TACI (transmembrane activator and calcium modulator and cyclophilin ligand interactor). The binding of each of the three receptors to BLyS plays a different role. The roles of BLyS in promoting the maturation of B cell in the transitional period of mice, prolonging the life span of mature B cells in mice and humans, and assisting in T cell activation are mainly achieved through BAFF-R. BCMA has a function of maintaining the survival of plasmablasts and promoting the antigen presentation of mature B cells. TACI receptors have a dual effect on the activation of mouse B cells. On the one hand, TACI-deficient mice exhibit increased peripheral B cells, increased antigen synthesis, autoimmunization and even lethal lymphoid proliferation, suggesting that TACI is an inhibitory receptor of BLyS. On the other hand, TACI participates in the immune response of T-cell independent antigen (TI). In addition, BAFF-R is a specific receptor for BLyS, while TACI and BCMA can also bind to APRIL in addition to binding to BLyS. Since TACI receptor has a strong affinity for both BLyS and APRIL, a drug based on the TACI receptor (e.g. TACI-Fc fusion protein) is superior in drug development compared to the other two receptors, and is preferred for drug structure.

TACI (transmembrane activator and CAML-interactor), a member of the tumor necrosis factor (TNF) receptor superfamily, is primarily expressed in B cells and has a weak expression in in monocytes, dendritic cells, and T cell lines. Currently, drugs based on the TACI receptor have been developed in two main types: TACI Fc fusion proteins as the predominant type and TACI antibodies. As of Sep. 2, 2022, only one TACI-Fc fusion protein has been approved for marketing globally, and two other drugs are in clinical trials.

| | Globally highest | |
|---|---|---|
| Drug | development stage | Indication |
| Telitacicept | Approved for | rheumatoid arthritis, Sjogren's |
| | marketing | syndrome, systemic lupus |
| | | erythematosus, multiple sclerosis, |
| | | myasthenia gravis, IgA |
| | | nephropathy, neuromyelitis optica |
| Atacicept | Phase II/III | rheumatoid arthritis, lupus |
| | | nephritis, systemic lupus |
| | | erythematosus, multiple |
| | | sclerosis, IgA nephropathy |
| Briobacept | Phase I | rheumatoid arthritis |

TACI Fc fusion proteins have shown promising therapeutic effects in autoimmune diseases. However, autoimmune diseases have complex mechanisms of action. In clinical practice, due to the lack of targeted companion diagnostics, patients are often treated with a step-by-step, trial-and-error approach with gradually enhanced response starting from a conventional dose. If the patient does not respond adequately, the researcher will apply an escalated dose. Therefore, in order to provide specific and personalized treatment regimens for patients with autoimmune diseases, it is necessary to supplement a deeper understanding of the disease manifestations in different patients with specific companion diagnostics. This means that the evaluation on absorption of drug in body fluid after administration of the patients, therapeutic efficacy monitoring, and prognosis assessment should be followed by providing an effective treatment regimen to ensure a correct, specific, and personalized treatment regimen for the patient.

Companion diagnostics (CDx) is an in vitro diagnostic technology related to targeted drugs, mainly by measuring the expression levels of the proteins and mutated genes in the human body to understand the therapeutic response of different patients to specific drugs, screen out the most appropriate medication population and treat them with targeted personalized treatment, to effectively improve their therapeutic efficiency and prognosis and reduce healthcare costs. On Aug. 6, 2014, the U.S. FDA issued Companion Diagnostics Guidelines. Companion diagnostics can help identify the patient population who are most likely to respond to therapeutic drugs, promote the use of drugs in a relatively limited market, and improve effectiveness and safety of drugs. In drug development, CDx facilitates the design of clinical trial in a small sample size to achieve clear and definite results with less investment during the development process. The advantage of companion diagnosis is that it can screen out effective treatment regimens for patients, save the time and cost of ineffective treatments, improve the medication adherence of patients, reduce the incidence of adverse reactions, and ensure drug safety and efficacy.

However, there is a lack of an antibody for the rapid and effective immunohistochemical detection of TACI expression, as well as a related detection method.

### SUMMARY

In order to overcome the defects of the prior art, the present disclosure provides an antibody capable of targeting TACI and a method for measuring the content of free TACI in a biological sample by using the TACI antibody. The measurement uses the content of TACI in a sample from a patient as a measurement index. The measured TACI content can provide a basis for prognosis assessment of a patient with an autoimmune disease and can also provide effective medication guidance for an autoimmune disease.

One embodiment of the present disclosure provides an antibody or antigen-binding fragment thereof, capable of binding specifically to TACI. Specifically, the antibody or antigen-binding fragment thereof provided by the present disclosure comprises:
(1) a heavy chain variable region (VH) comprising VH-CDR1 having an amino acid sequence of SEQ ID NO: 1, VH-CDR2 having an amino acid sequence of SEQ ID NO: 2 and VH-CDR3 having an amino acid sequence of SEQ ID NO: 3 (Kabat numbering, the same for the following description),

SEQ ID NO: 1
EFSMH
SEQ ID NO: 2
GINPNHGGSSYNQKFKG
SEQ ID NO: 3
RGDYYGNLYYAMDY

(2) a heavy chain variable region (VH) comprising VH-CDR1 having an amino acid sequence of SEQ ID NO: 4, VH-CDR2 having an amino acid sequence of SEQ ID NO: 5 and VH-CDR3 having an amino acid sequence of SEQ ID NO: 6, or

SEQ ID NO: 4
AYALH
SEQ ID NO: 5
GINPNHGGVSYNQKFKD
SEQ ID NO: 6
FRWDLYYGMDY

(3) a heavy chain variable region (VH) comprising VH-CDR1 having an amino acid sequence of SEQ ID NO: 7, VH-CDR2 having an amino acid sequence of SEQ ID NO: 8 and VH-CDR3 having an amino acid sequence of SEQ ID NO: 9; and/or

SEQ ID NO: 7
EYAMH
SEQ ID NO: 8
GINPNHGGTSNNQKFKG
SEQ ID NO: 9
WGTDPFFAY

(4) a light chain variable region (VL) comprising VL-CDR1 having an amino acid sequence of SEQ ID NO: 10, VL-CDR2 having an amino acid sequence of SEQ ID NO: 11 and VL-CDR3 having an amino acid sequence of SEQ ID NO: 12, or

SEQ ID NO: 10
KASQSVSNDVV
SEQ ID NO: 11
YASNRYT
SEQ ID NO: 12
QQDYSSPWA

(5) a light chain variable region (VL) comprising VL-CDR1 having an amino acid sequence of SEQ ID NO: 13, VL-CDR2 having an amino acid sequence of SEQ ID NO: 14 and VL-CDR3 having an amino acid sequence of SEQ ID NO: 15, or

SEQ ID NO: 13
SDNSNMIYMY
SEQ ID NO: 14
DTSRLAS
SEQ ID NO: 15
HQRSIYSWT

(6) a light chain variable region (VL) comprising VL-CDR1 having an amino acid sequence of SEQ ID NO: 16, VL-CDR2 having an amino acid sequence of SEQ ID NO: 17 and VL-CDR3 having an amino acid sequence of SEQ ID NO: 18.

SEQ ID NO: 16
KASQDVSTAVA
SEQ ID NO: 17
WASTRHT
SEQ ID NO: 18
HQHYAPPWT

Further, the antibody is a mouse antibody.

Further, the antibody is a rabbit antibody.

Further, the antibody is a murine-derived antibody.

Further, the antibody is a rabbit-derived antibody.

Further, the antigen-binding fragment is Fab.

Further, the antigen-binding fragment is F(ab′)₂.

Further, the antigen-binding fragment is Fab′.

Further, the antigen-binding fragment is Fv.

Further, the antigen-binding fragment is ScFv.

Further, the antibody or antigen binding fragment thereof comprises:
(1) a heavy chain variable region (VH), wherein the heavy chain variable region has at least 95%, 96%, 97%, 98%, 99% or 100% sequence identity to the amino acid sequence set forth in SEQ ID NO: 19, SEQ ID NO: 20 or SEQ ID NO: 21, and/or

| SEQ ID NO: 19 | |
|---|---|
| MGWSWIFLFL LSGTAGVLSE VQLQQSGPEL VKPGASVKIS CKTSGYTFTE FSMHWVQQSH | 60 |
| GKSLEWIGGI NPNHGGSSYN QKFKGKATLT VDKSSSTAYM ELRSLTSEDS AVYYCARRGD | 120 |
| YYGNLYYAMD YWGQGTSVTV SS | 142 |
| SEQ ID NO: 20 | |
| MGWSWIFLFL LSGTAGVLSE VQLQQSGPEL VRPGASVKKS CKTSGYTFTA YALHWVKQSH | 60 |
| GTSLEWIGGI NPNHGGVSYN QKFKDKATLT VDKSSSTAYM ELRSLTSEDS AVYYCARFRW | 120 |
| DLYYGMDYWG QGTSVTVAS | 139 |
| SEQ ID NO: 21 | |
| MGWSWIFLFL LSGTAGVLSE VQLQQSGPEL VKPGASVKIS CKTSGYTFTE YAMHWVKQSH | 60 |
| GKSLEWIGGI NPNHGGTSNN QKFKGKATLT VDKSSSTAYM DFRSLTSEDS AVYYCARWGT | 120 |
| DPFFAYWGQG TLVTVSA | 137 |

(2) a light chain variable region (VL), wherein the light chain variable region has at least 95%, 96%, 97%, 98%, 99% or 100% sequence identity to the amino acid sequence set forth in SEQ ID NO: 22, SEQ ID NO: 23 or SEQ ID NO: 24.

| SEQ ID NO: 22 | |
|---|---|
| MKSQTQVFVF LLLCVSGAHG SIVMTQTPKF LLVSVGDSIT ITCKASQSVS NDVVWYQQKP | 60 |
| GQSPKLLIYY ASNRYTGVPD RFTGSGYGTD FTFTITTVQA EDLAVYFCQQ DYSSPWAFGG | 120 |
| GTKLEIKRA | 129 |
| SEQ ID NO: 23 | |
| MDFQVQIFSF LLISASVILS RGQIVLTQSP AIISVSSGEK VTMPCSDNSN MIYMYWNLWK | 60 |
| PGTSPKRWIY DTSRLASGVP ARFSGSGFGT SFSLTVSSME AEDAATYYCH QRSIYSWTFG | 120 |
| GGTKLEIKRA | 130 |
| SEQ ID NO: 24 | |
| MESQIQAFVF VFLWLSGVDG DIVMTQSHKF MSTSVGDRVS ITCKASQDVS TAVAWYQQKP | 60 |
| GQSPKLLIYW ASTRHTGVPD RFTGSGSGTD YTLTITSVQA EDLTLYYCHQ HYAPPWTFGG | 120 |
| GTKLEIKRA | 129 |

Still further, the antibody or antigen binding fragment thereof comprises:
(1) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 19, and the light chain variable region has at least 95%, 96%, 97%, 98%, 99% or 100% sequence identity to the amino acid sequence set forth in SEQ ID NO: 22, or
(2) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 20, and the light chain variable region has at least 95%, 96%, 97%, 98%, 99% or 100% sequence identity to the amino acid sequence set forth in SEQ ID NO: 23, or
(3) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 21, and the light chain variable region has at least 95%, 96%, 97%, 98%, 99% or 100% sequence identity to the amino acid sequence set forth in SEQ ID NO: 24.

In some embodiments, the antibody or antigen binding fragment thereof comprises a heavy variable region (VH) having an amino acid sequence set forth in SEQ ID NO: 19 and/or a light variable region (VL) having an amino acid sequence set forth in SEQ ID NO: 22.

In some embodiments, the antibody or antigen binding fragment thereof comprises a heavy variable region (VH) having an amino acid sequence set forth in SEQ ID NO: 20 and/or a light variable region (VL) having an amino acid sequence set forth in SEQ ID NO: 23.

In some embodiments, the antibody or antigen binding fragment thereof comprises a heavy variable region (VH) having an amino acid sequence set forth in SEQ ID NO: 21 and/or a light variable region (VL) having an amino acid sequence set forth in SEQ ID NO: 24.

Further, the antibody or antigen binding fragment thereof comprises an Fc region.

Still further, the Fc region comprises an amino acid sequence having at least 95%, 96%, 97%, 98%, 99%, or 100% sequence identity to the amino acid sequence set forth in SEQ ID NO: 25, SEQ ID NO: 26 or SEQ ID NO: 27.

| SEQ ID NO: 25 | |
|---|---|
| AKTTPPSVYP LAPGSAAQTN SMVTLGCLVK GYFPEPVTVT WNSGSLSSGV HTFPAVLQSD | 60 |
| LYTLSSSVTV PSSTWPSETV TCNVAHPASS TKVDKKIVPR DCGCKPCICT VPEVSSVFIF | 120 |
| PPKPKDVLTI TLTPKVTCVV VDISKDDPEV QFSWFVDDVE VHTAQTQPRE EQFNSTFRSV | 180 |
| SELPIMHQDW LNGKEFKCRV NSAAFPAPIE KTISKTKGRP KAPQVYTIPP PKEQMAKDKV | 240 |
| SLTCMITDFF PEDITVEWQW NGQPAENYKN TQPIMDTDGS YFVYSKLNVQ KSNWEAGNTF | 300 |
| TCSVLHEGLH NHHTEKSLSH SPGK | 324 |
| SEQ ID NO: 26 | |
| AKTTPPSVYP LAPGSAAQTN SMVTLGCLVK GYFPEPVTVT WNSGSLSSGV HTFPAVLQSD | 60 |
| LYTLSSSVTV PSSTWPSETV TCNVAHPASS TKVDKKIVPR DCGCKPCICT VPEVSSVFIF | 120 |
| PPKPKDVLTI TLTPKVTCVV VDISKDDPEV QFSWFVDDVE VHTAQTQPRE EQFNSTFRSV | 180 |
| SELPIMHQDW LNGKEFKCRV NSAAFPAPIE KTISKTKGRP KAPQVYTIPP PKEQMAKDKV | 240 |
| SLTCMITDFF PEDITVEWQW NGQPAENYKN TQPIMDTDGS YFVYSKLNVQ KSNWEAGNTF | 300 |
| TCSVLHEGLH NHHTEKSLSH SPGK | 324 |
| SEQ ID NO: 27 | |
| AKTTPPSVYP LAPGSAAQTN SMVTLGCLVK GYFPEPVTVT WNSGSLSSGV HTFPAVLQSD | 60 |
| LYTLSSSVTV PSSTWPSETV TCNVAHPASS TKVDKKIVPR DCGCKPCICT VPEVSSVFIF | 120 |
| PPKPKDVLTI TLTPKVTCVV VDISKDDPEV QFSWFVDDVE VHTAQTQPRE EQFNSTFRSV | 180 |
| SELPIMHQDW LNGKEFKCRV NSAAFPAPIE KTISKTKGRP KAPQVYTIPP PKEQMAKDKV | 240 |
| SLTCMITDFF PEDITVEWQW NGQPAENYKN TQPIMDTDGS YFVYSKLNVQ KSNWEAGNTF | 300 |
| TCSVLHEGLH NHHTEKSLSH SPGK | 324 |

In some embodiments, the Fc region has an amino acid sequence set forth in SEQ ID NO: 25.

In some embodiments, the Fc region has an amino acid sequence set forth in SEQ ID NO: 26.

In some embodiments, the Fc region has an amino acid sequence set forth in SEQ ID NO: 27.

Further, the antibody or antigen-binding fragment thereof comprises:
(1) a heavy chain, wherein the heavy chain has at least 95%, 96%, 97%, 98%, 99%, or 100% sequence identity to the amino acid sequence set forth in SEQ ID NO: 28, SEQ ID NO: 29 or SEQ ID NO: 30; and/or

| SEQ ID NO: 28 | |
|---|---|
| MGWSWIFLFL LSGTAGVLSE VQLQQSGPEL VKPGASVKIS CKTSGYTFTE FSMHWVQQSH | 60 |
| GKSLEWIGGI NPNHGGSSYN QKFKGKATLT VDKSSSTAYM ELRSLTSEDS AVYYCARRGD | 120 |
| YYGNLYYAMD YWGQGTSVTV SSAKTTPPSV YPLAPGSAAQ TNSMVTLGCL VKGYFPEPVT | 180 |
| VTWNSGSLSS GVHTFPAVLQ SDLYTLSSSV TVPSSTWPSE TVTCNVAHPA SSTKVDKKIV | 240 |
| PRDCGCKPCI CTVPEVSSVF IFPPKPKDVL TITLTPKVTC VVVDISKDDP EVQFSWFVDD | 300 |
| VEVHTAQTQP REEQFNSTFR SVSELPIMHQ DWLNGKEFKC RVNSAAFPAP IEKTISKTKG | 360 |
| RPKAPQVYTI PPPKEQMAKD KVSLTCMITD FFPEDITVEW QWNGQPAENY KNTQPIMDTD | 420 |
| GSYFVYSKLN VQKSNWEAGN TFTCSVLHEG LHNHHTEKSL SHSPGK | 466 |
| SEQ ID NO: 29 | |
| MGWSWIFLFL LSGTAGVLSE VQLQQSGPEL VRPGASVKKS CKTSGYTFTA YALHWVKQSH | 60 |
| GTSLEWIGGI NPNHGGVSYN QKFKDKATLT VDKSSSTAYM ELRSLTSEDS AVYYCARFRW | 120 |
| DLYYGMDYWG QGTSVIVASA KTTPPSVYPL APGSAAQTNS MVTLGCLVKG YFPEPVTVTW | 180 |
| NSGSLSSGVH TFPAVLQSDL YTLSSSVTVP SSTWPSETVT CNVAHPASST KVDKKIVPRD | 240 |
| CGCKPCICTV PEVSSVFIFP PKPKDVLTIT LTPKVTCVVV DISKDDPEVQ FSWFVDDVEV | 300 |
| HTAQTQPREE QFNSTFRSVS ELPIMHQDWL NGKEFKCRVN SAAFPAPIEK TISKTKGRPK | 360 |
| APQVYTIPPP KEQMAKDKVS LTCMITDFFP EDITVEWQWN GQPAENYKNT QPIMDTDGSY | 420 |
| FVYSKLNVQK SNWEAGNTFT CSVLHEGLHN HHTEKSLSHS PGK | 463 |
| SEQ ID NO: 30 | |
| MGWSWIFLFL LSGTAGVLSE VQLQQSGPEL VKPGASVKIS CKTSGYTFTE YAMHWVKQSH | 60 |
| GKSLEWIGGI NPNHGGTSNN QKFKGKATLT VDKSSSTAYM DFRSLTSEDS AVYYCARWGT | 120 |
| DPFFAYWQG TLVTVSAAKT TPPSVYPLAP GSAAQTNSMV TLGCLVKGYF PEPVTVTWNS | 180 |
| GSLSSGVHTF PAVLQSDLYT LSSSVTVPSS TWPSETVTCN VAHPASSTKV DKKIVPRDCG | 240 |
| CKPCICTVPE VSSVFIFPPK PKDVLTITLT PKVTCVVVDI SKDDPEVQFS WFVDDVEVHT | 300 |
| AQTQPREEQF NSTFRSVSEL PIMHQDWLNG KEFKCRVNSA AFPAPIEKTI SKTKGRPKAP | 360 |
| QVYTIPPPKE QMAKDKVSLT CMITDFFPED ITVEWQWNGQ PAENYKNTQP IMDTDGSYFV | 420 |
| YSKLNVQKSN WEAGNTFTCS VLHEGLHNHH TEKSLSHSPG K | 461 |

(2) a light chain, wherein the light chain has at least 95%, 96%, 97%, 98%, 99%, or 100% sequence identity to the amino acid sequence set forth in SEQ ID NO: 31, SEQ ID NO: 32 or SEQ ID NO: 33.

| SEQ ID NO: 31 | |
|---|---|
| MKSQTQVFVF LLLCVSGAHG SIVMTQTPKF LLVSVGDSIT ITCKASQSVS NDVVWYQQKP | 60 |
| GQSPKLLIYY ASNRYTGVPD RFTGSGYGTD FTFTITTVQA EDLAVYFCQQ DYSSPWAFGG | 120 |
| GTKLEIKRAD AAPTVSIFPP SSEQLTSGGA SVVCFLNNFY PKDINVKWKI DGSERQNGVL | 180 |
| NSWTDQDSKD STYSMSSTLT LTKDEYERHN SYTCEATHKT STSPIVKSFN RNEC | 234 |
| SEQ ID NO: 32 | |
| MDFQVQIFSF LLISASVILS RGQIVLTQSP AIISVSSGEK VTMPCSDNSN MIYMYWNLWK | 60 |
| PGTSPKRWIY DTSRLASGVP ARFSGSGFGT SFSLTVSSME AEDAATYYCH QRSIYSWTFG | 120 |
| GGTKLEIKRA DAAPTVSIFP PSSEQLTSGG ASVVCFLNNF YPKDINVKWK IDGSERQNGV | 180 |
| LNSWTDQDSK DSTYSMSSTL TLTKDEYERH NSYTCEATHK TSTSPIVKSF NRNEC | 235 |
| SEQ ID NO: 33 | |
| MESQIQAFVF VFLWLSGVDG DIVMTQSHKF MSTSVGDRVS ITCKASQDVS TAVAWYQQKP | 60 |
| GQSPKLLIYW ASTRHTGVPD RFTGSGSGTD YTLTITSVQA EDLTLYYCHQ HYAPPWTFGG | 120 |
| GTKLEIKRAD AAPTVSIFPP SSEQLTSGGA SVVCFLNNFY PKDINVKWKI DGSERQNGVL | 180 |
| NSWTDQDSKD STYSMSSTLT LTKDEYERHN SYTCEATHKT STSPIVKSFN RNEC | 234 |

Further, the antibody or antigen binding fragment thereof comprises:
(1) a heavy chain and/or a light chain, wherein the heavy chain has at least 95%, 96%, 97%, 98%, 99% or 100% sequence identity to the amino acid sequence set forth in SEQ ID NO: 28, and the light chain has at least 95%, 96%, 97%, 98%, 99% or 100% sequence identity to the amino acid sequence set forth in SEQ ID NO: 31; or
(2) a heavy chain and/or a light chain, wherein the heavy chain has at least 95%, 96%, 97%, 98%, 99% or 100% sequence identity to the amino acid sequence set forth in SEQ ID NO: 29, and the light chain has at least 95%, 96%, 97%, 98%, 99% or 100% sequence identity to the amino acid sequence set forth in SEQ ID NO: 32; or
(3) a heavy chain and/or a light chain, wherein the heavy chain has at least 95%, 96%, 97%, 98%, 99% or 100% sequence identity to the amino acid sequence set forth in SEQ ID NO: 30, and the light chain has at least 95%, 96%, 97%, 98%, 99% or 100% sequence identity to the amino acid sequence set forth in SEQ ID NO: 33.

In some embodiments, the antibody or antigen binding fragment thereof comprises a heavy chain having an amino acid sequence set forth in SEQ ID NO: 28 and/or a light chain having an amino acid sequence set forth in SEQ ID NO: 31.

In some embodiments, the antibody or antigen binding fragment thereof comprises a heavy chain having an amino acid sequence set forth in SEQ ID NO: 29 and/or a light chain having an amino acid sequence set forth in SEQ ID NO: 32.

In some embodiments, the antibody or antigen binding fragment thereof comprises a heavy chain having an amino acid sequence set forth in SEQ ID NO: 30 and/or a light chain having an amino acid sequence set forth in SEQ ID NO: 33.

Further, the antibody or antigen-binding fragment thereof can be further conjugated with a detectable label.

Still further, the detectable label is an enzyme.

Still further, the detectable label is a light-emitting label.

Still further, the detectable label is a radioisotope.

Still further, the detectable label is a chromogenic label.

Still further, the detectable label is a hapten.

Still further, the detectable label is a metal complex.

One embodiment of the present disclosure further provides use of the antibody or antigen-binding fragment thereof above in the measurement of TACI content in a sample.

Further, the sample is a biological sample.

Still further, the sample is a blood sample.

Further, the sample is derived from a subject overexpressing BLyS and APRIL.

Still further, the sample is derived from a subject with an autoimmune disease.

Still further, the autoimmune disease is one or more selected from the group consisting of rheumatic arthritis, rheumatoid arthritis, juvenile rheumatoid arthritis, systemic lupus erythematosus (SLE), lupus nephritis (LN), Wegener's disease, inflammatory bowel disease, idiopathic thrombocytopenia purpura (ITP), thrombotic thrombocytopenia purpura (TTP), autoimmune thrombocytopenia, multiple sclerosis, psoriasis, IgA nephropathy, IgM polyneuropathy, myasthenia gravis, vasculitis, diabetes mellitus, Raynaud's syndrome, Sjogren's syndrome, glomerular nephritis, autoimmune hepatitis, autoimmune thyroiditis, neuromyelitis optica and sicca syndrome.

In some embodiments, the autoimmune disease is rheumatic arthritis. In some other embodiments, the autoimmune disease is rheumatoid arthritis. In some other embodiments, the autoimmune disease is juvenile rheumatoid arthritis. In some other embodiments, the autoimmune disease is systemic lupus erythematosus (SLE). In some other embodiments, the autoimmune disease is lupus nephritis (LN). In some other embodiments, the autoimmune disease is Wegener's disease. In some other embodiments, the autoimmune disease is inflammatory bowel disease. In some other embodiments, the autoimmune disease is idiopathic thrombocytopenia purpura (ITP). In some other embodiments, the autoimmune disease is thrombotic thrombocytopenia purpura (TTP). In some other embodiments, the autoimmune disease is autoimmune thrombocytopenia. In some other embodiments, the autoimmune disease is multiple sclerosis. In some other embodiments, the autoimmune disease is psoriasis. In some other embodiments, the autoimmune disease is IgA nephropathy. In some other embodiments, the autoimmune disease is IgM polyneuropathy. In some other embodiments, the autoimmune disease is myasthenia gravis. In some other embodiments, the autoimmune disease is vasculitis. In some other embodiments, the autoimmune disease is diabetes mellitus. In some other embodiments, the autoimmune disease is Raynaud's syndrome. In some other embodiments, the autoimmune disease is Sjogren's syndrome. In some other embodiments, the autoimmune disease is glomerular nephritis. In some other embodiments, the autoimmune disease is autoimmune hepatitis. In some other embodiments, the autoimmune disease is autoimmune thyroiditis. In some other embodiments, the autoimmune disease is neuromyelitis optica. In some other embodiments, the autoimmune disease is sicca syndrome.

In some other embodiments, the autoimmune disease is an overlap syndrome involving two, three, four or even more of the above diseases. That is, the patient has concurrent manifestations of several autoimmune diseases. By way of non-limiting example, the autoimmune disease is an overlap syndrome manifested by rheumatoid arthritis and systemic lupus erythematosus, and the autoimmune disease is manifested as rheumatoid arthritis, systemic lupus erythematosus (SLE) and multiple sclerosis.

Still further, the subject is a human or a mammal. In one embodiment, the subject is a human.

Further, the measurement is carried out by ELISA.

Further, the measurement is carried out by Western blot.

Further, the measurement is carried out by immunohistochemistry (IHC).

One embodiment of the present disclosure provides a method for measuring TACI content in a sample, wherein the method comprises the following steps: contacting a sample with the above-mentioned antibody or antigen-binding fragment thereof, and detecting the existence of the bound antibody or antigen-binding fragment thereof.

In some embodiments, for the method of measuring TACI content in a sample provided by the present disclosure, the method comprises the following exemplary steps:
(1) coating: coating the above-mentioned TACI antibody or antigen-binding fragment thereof onto a 96-well microplate and incubating at a specific temperature,
(2) blocking: blocking with a blocking solution and incubating,
(3) contacting with a sample: adding standards, a quality control sample, a sample to be tested and a blank control, incubating for complete binding of free TACI in the sample to the TACI antibody (1B11D2),
(4) binding of secondary antibody: adding the secondary antibody,
(5) color development of secondary antibody: adding a color developing agent,
(6) color development of substrate: adding a substrate for color development,
(7) terminating: adding a stop solution to terminate the reaction, and
(8) reading the difference of values at 450 nm and 570 nm by using a microplate reader.

One embodiment of the present disclosure further provides an isolated polynucleotide encoding the above-mentioned antibody or antigen-binding fragment thereof.

One embodiment of the present disclosure further provides an expression vector comprising the above-mentioned polynucleotide.

One embodiment of the present disclosure further provides use of the expression vector in the manufacture of the above-mentioned antibody or antigen-binding fragment thereof.

One embodiment of the present disclosure further provides a host cell comprising at least one expression vector described above.

One embodiment of the present disclosure further provides use of the above-mentioned antibody or antigen-binding fragment thereof in the manufacture of a kit.

One embodiment of the present disclosure further provides a kit comprising the above-mentioned antibody.

The present disclosure provides an antibody targeting TACI or an antigen binding fragment thereof. The TACI antibody is capable of measuring the TACI content in a patient, particularly the content of free TACI or TACI-Fc fusion protein drugs. Thus, the level of drug absorption in a patient can be calculated according to the level of drug in the patient, thereby playing a role in assessing the condition of the patient, monitoring therapeutic efficacy and assessing prognosis.

### BRIEF DESCRIPTION OF DRAWINGS

FIG. 1 is a butterfly plot analyzing the deuteration consistency between control group B-1 and interaction group B.

FIG. 2 is a residue plot showing deuteration difference between control group B-1 and interaction group B.

FIG. 3 is a residue plot showing deuteration difference percentage between control group B-1 and interaction group B.

FIG. 4 is a butterfly plot analyzing the deuteration consistency between control group A-1 and interaction group A.

FIG. 5 is a residue plot showing deuteration difference between control group A-1 and interaction group A.

FIG. 6 is a residue plot showing deuteration difference percentage between control group A-1 and interaction group A.

FIG. 7 is a standard curve of OD value versus free telitacicept.

### DETAILED DESCRIPTION

### Definitions

Unless otherwise defined, all terms used herein have the same meaning as understood in the art. For definitions and terms in the art, reference can be made a reference to Current Protocols in Molecular Biology (Ausubel). The abbreviations for amino acid residues are the standard 3-letter and/or 1-letter codes used in the art to refer to one of the 20 commonly used L-amino acids.

The term “TACI” refers to Transmembrane Activator and CAML Interactor. Typically, wild-type TACI is a type III membrane protein comprising 293 amino acid residues (166 residues in the extracellular functional region, 10 residues in the transmembrane region and 117 residues in the intracellular functional region), with no signal peptide in the amino-terminal. There are two cysteine-rich repeat regions (S33-66 and C70-C104) in the extracellular region, which belong to the TNFR (tumor necrosis factor receptor) superfamily but lack the death domain of the TNF family compared to other members of the TNF (tumor necrosis factor) receptor superfamily.

The term “antibody” is used in the broadest scope and encompasses various antibody structures including, but not limited to, a monoclonal antibody, a polyclonal antibody, a multispecific antibody (e.g., bispecific antibody), and an antibody fragment. In particular, “antibody” as used herein refers to a protein comprising at least two heavy chains and two light chains interconnected by disulfide bonds. Each heavy chain comprises a heavy chain variable region (one-fifth or one-quarter region on the heavy chain near the N-terminal) and a heavy chain constant region (three-quarters or four-fifths region on the heavy chain near the C-terminal). Each light chain comprises a light chain variable region (a half region on the light chain near the N-terminal) and a light chain constant region (a half region on the light chain near the C-terminal). The variable region of the heavy chain and the variable region of the light chain can be further subdivided into multiple regions with high variability, which is called a complementary determining region (CDR). The “CDR” used herein refers to the hypervariable regions of the heavy and light chains of immunoglobulins, including those determined by the Kabat, Chothia, IMGT, AbM and Contact systems. There are three heavy chain CDRs and three light chain CDRs for each antibody. Depending on the circumstances, the term CDR as used herein is intended to indicate one or several or even all of these regions, which comprise most of the amino acid residues responsible for binding through the affinity of an antibody to an antigen or a recognition epitope thereof.

The term “antigen-binding fragment” refers to an antibody fragment comprising heavy chain variable region or a light chain variable region of an antibody. The fragment is sufficient to retain the same binding specificity as the antibody from which it is derived and a sufficient affinity, particularly including, but not limited to, a Fab fragment, a Fab′ fragment, a F (ab) 2 fragment, a F(ab′) 2 fragment, a Fv fragment, a scFv fragment and/or an isolated complementarity determining region. In the present disclosure, the term “Fab” generally refers to a monovalent fragment consisting of VH, VL, CL and CH1 domains. The term Fab′ generally refers to fragments which differ from Fab by the addition of a few residues (including one or more cysteine residues from the antibody hinge region) at the carboxyl terminus of the heavy chain CH1 domain. The term “F(ab′) 2” generally refers to a dimer of Fab′, which is a bivalent fragment comprising two Fab fragments linked by a disulfide bond at the hinge region. The term “Fv” generally refers to the Fv fragment consisting of the VL and VH domains of a single arm of an antibody. The term “scFv” generally refers to a monovalent molecule formed by connection and pairing of VH and VL through a linker; such scFv molecule can have the general structure: NH₂-VL-linker-VH—COOH or NH₂—VH-linker-VL-COOH. These antibody fragments are obtained using conventional techniques known in the art, and the fragments can be screened for utility in the same manner as intact antibodies.

The term “mouse antibody” refers to an antibody having heavy and light chains only derived from mouse B cells. This antibody thus consists of mouse amino acid sequences, regardless of the origin of the cells from which it is produced.

The term “rabbit antibody” refers to an antibody having variable and constant regions or a domain substantially corresponding to the rabbit germ line immunoglobulin sequences known in the art.

The terms “murine-derived antibody” and “rabbit-derived antibody” used herein refer to monoclonal antibodies produced according to knowledge and skill in the art. During the production, the test subject is injected with the corresponding antigen, and then the hybridomas expressing the antibody with the desired sequence or functional property are isolated. In one embodiment of the present disclosure, the murine- or rabbit-derived antibodies or antigen-binding fragment thereof may further comprise a light chain constant region of murine-derived or rabbit-derived κ, λ chain or a variant thereof, or further comprise a heavy chain constant region of murine-derived or rabbit-derived IgG1, IgG2, IgG3, or a variant thereof.

The term “Fc region” defines a C-terminal region of an immunoglobulin heavy chain, including, for example, native sequence Fc regions, recombinant Fc regions and variant Fc regions. Although the boundaries of the Fc region of an immunoglobulin heavy chain may vary slightly, the Fc region of a human IgG heavy chain is typically defined to stretch from an amino acid residue at position Cys226 or from Pro230 to the carboxyl terminus thereof. The C-terminal lysine of the Fc region (residue 447 according to the EU numbering system) may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with a K447 residue removed, and antibody populations having a mixture of antibodies with and without the K447 residue. A functional “Fc region” has an “effector function” of a native sequence Fc region. Exemplary “effector functions” include: C1q binding, CDC (complement dependent cytotoxicity), Fc receptor binding, ADCC (antibody-dependent cell-mediated cytotoxicity), phagocytosis and down regulation of cell surface receptors (e.g. B cell receptor). Such effector functions typically require binding of the Fe region to a binding region or binding domain (e.g., a variable region or domain of an antibody) and can be assessed using a variety of assays known in the art. A variant “Fe region” comprises an amino acid sequence that differs from the amino acid sequence of the native sequence Fc region due to at least one amino acid modification (e.g., substitution, addition, or deletion). In some embodiments, the variant Fc region has at least one amino acid substitution, e.g., about 1 to about 10 amino acid substitutions, or about 1 to about 5 amino acid substitutions, in the native sequence Fc region or in the Fe region of the parent polypeptide, as compared to the native sequence Fe region or as compared to the Fc region of the parent polypeptide. The variant Fe region herein may have at least about 80%, or have at least 90% homology, for example at least about 95% homology, with the native sequence Fc region and/or with the Fc region of the parent polypeptide.

The term “detectable label” refers to any component capable of providing a detection signal under a detection condition, and comprises directly and indirectly detectable labels. Detectable labels useful in the methods described herein comprise any component that can be detected indirectly or directly by spectroscopic, photochemical, biochemical, immunochemical, electrical, optical, chemical or other means. Examples thereof include: antigen label (e.g., digoxigenin (DIG), fluorescein or dinitrophenol (DNP) and the like), biotin capable of being stained with labeled streptavidin conjugate, fluorescent dye (e.g., fluorescein, Texaco red, rhodamine or fluorophore label such as Alex Fluor label and the like), radioactive label (e.g., ¹²⁵I, ³⁵S, ¹⁴C or ³²P), enzyme (e.g., Streptavidin-HRP, peroxidase, alkaline phosphatase, galactase and other enzymes commonly used in ELISA), fluorescent protein (e.g., green fluorescent protein, red fluorescent protein, yellow fluorescent protein and the like), synthetic metal-chelating polymer (metallo-complex) and colorimetric label.

The term “enzyme” refers to a protein molecule that can catalyze a biochemical reaction in metabolism occurred in a cell or extracellular medium. Specific examples include oxidoreductases (such as oxidases, reductases, peroxidases, oxygenases, hydrogenases or dehydrogenases), transferases (such as kinases, transaminases and translocases), hydrolases (such as esterases, peptidases, glycosidases and glucosidases), lyases (such as decarboxylases, aldolases and dehydrases), isomerases (such as racemase and epimerases) and ligases. In one embodiment, the enzyme is a peroxidase, more preferably a Streptavidin-HRP enzyme.

The term “light-emitting label” refers to a label that can emit light under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminescence. An external excitation can be a combination of the above.

The term “hapten” refers to a partial or incomplete antigen. A hapten is a protein-free substance which is not capable of stimulating antibody formation but reacts with antibodies.

The term “metal complex” means a metal-containing compound, which contains a metal atom or ion and a surrounding array of bound molecules or ions (known as ligands).

The term “biological sample” includes, but is not limited to, any quantity of a substance from a living organism or formerly living organism. Such living organisms include, but are not limited to, humans, mice, monkeys, rats, rabbits and other animals. Such substances include, but are not limited to, blood, serum, urine, cells, organs, tissues, bone, bone marrow, lymph nodes and skin.

The term “blood sample” refers to any sample prepared from blood, such as plasma and blood cells separated from blood.

The term “BLyS” includes those described in J Leukoc Biol. 1999, 65:680 (Shu et al.); GenBank Accession: No. AF136293; WO98/18921 published on May 7, 1998; EP86/9180 published on Oct. 7, 1998; WO98/27114 published on Jun. 25, 1998; WO99/12964 published on Mar. 18, 1999; WO99/33980 published on Jul. 8, 1999; Moore et al., Science, 285:260-263 (1999); Schneider et al., J. Exp. Med., 189:1747-1756 (1999) and Mukhopadhyay et al., J. Biol. Chem., 274:15978-15981 (1999).

The term “APRIL” refers to A proliferation-inducing ligand consisting of 184 amino acid residues (NCBI Reference Sequence: NP_003799.1), which belongs to the TNF superfamily.

The term “autoimmune disease” refers to a disease in which the immune system produces an abnormal immune response compared to a healthy population. Examples of autoimmune diseases include, but are not limited to, acute disseminated encephalomyelitis, acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome, autoimmune angioedema, autoimmune aplastic anemia, autoimmune dysautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenia purpura, autoimmune thyroid disease, autoimmune urticarial, axonal or neuronal neuropathies, Balo disease, Behcet's disease, cardiomyopathy, Castleman disease, Chagas disease, chronic fatigue syndrome, chronic inflammatory demyelinating polyneuropathy, chronic recurrent multifocal osteomyelitis (CRMO), Churg-Strauss syndrome, cicatricial pemphigoid/benign mucosal pemphigoid, Cogan's syndrome, cold agglutinin disease, congenital heart block, coxsackie myocarditis, CREST disease, essential mixed cryoglobulinemia, demyelinating neuropathies, dermatitis herpetiformis, dermatomyositis, neuromyelitis optica, discoid lupus, Dressler's syndrome, endometriosis, eosinophilic esophagitis, eosinophilic fasciitis, erythema nodosum, experimental allergic encephalomyelitis, Evan's syndrome, fibromyalgia, fibrosing alveolitis, giant cell arteritis, giant cell myocarditis, glomerular nephritis, Good pasture's syndrome, granulomatosis with polyangiitis, Grave's disease, Guillain-Barre syndrome, hemolytic anemia, Henock-Schonlein purpura, herpes gestationis, hypogammaglobulinemia, idiopathic thrombocytopenia purpura, IgA nephropathy, IgG4-related sclerosing diseases, immunoregulatory lipoprotein disease, inclusion body myositis, interstitial cystitis, juvenile myositis, Kawasaki syndrome, Lambert-Eaton syndrome, leukocytoclastic vasculitis, lichen planus, lichen sclerosus, ligneous conjunctivitis, linear IgA disease, Lyme disease, choric Meniere's disease, microscopic polyangitis, mixed connective tissue disease, Mooren's ulcer, Mucha-Habermann disease, multiple sclerosis, myasthenia gravis, myositis, narcolepsy, neutropenia, ocular cicatricial pemphigoid, optic neuritis, palindromic rheumatism, paraneoplastic cerebellar degeneration, paroxysmal nocturnal hemoglobinuria, Parry Romberg syndrome, Parsonage-Turner syndrome, pars planitis, pemphigus, peripheral neuropathy, perivenous encephalomyelitis, pernicious anemia, POEMS syndrome, polyarteritis nodosa, type I, II and III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, idiopathic pulmonary fibrosis, pyoderma gangrenosum, pure red cell aplasia, Raynaud's phenomena, reactive arthritis, reflex sympathetic dystrophy, Reiter's syndrome, relapsing polychondritis, restless legs syndrome, retroperitoneal fibrosis, rheumatic fever, rheumatic arthritis, rheumatoid arthritis, juvenile rheumatoid arthritis, sarcoidosis, Schmidt syndrome, scleritis, scleroderma. sperm and testicular autoimmunity, stiff person syndrome, subacute bacterial endocarditis, Susac's syndrome, sympathetic ophthalmia, Takayasu's arteritis, temporal arteritis/giant cell arteries, Tolosa-Hunt syndrome, transverse myelitis, undifferentiated connective tissue disease, uveitis, vasculitis, vesiculobullous dermatosis, vitiligo or Wegener's granulomatosis, systemic lupus erythematosus (SLE), Lupus nephritis (LN), Wegener's disease, inflammatory bowel disease, thrombotic thrombocytopenia purpura (TTP), autoimmune thrombocytopenia, psoriasis, IgM polyneuropathy, angiitis, diabetes mellitus, Raynaud's syndrome, Sjogren's syndrome, glomerular nephritis, autoimmune hepatitis, autoimmune thyroiditis, and sicca syndrome. The concurrent manifestations of two, three, four, or even more of these diseases are referred to as “overlap syndromes”.

The term “ELISA” refers to a qualitative and quantitative assay in which a soluble antigen or antibody is attached to a solid phase support (e.g. polystyrene) for an immunologic reaction based on antigen-antibody binding specificity.

The term “Western blot” includes not only the standard western blot, but also various variants such as Far-Western blot, Northwestern blot and Southwestern blot. In general, a western blot involves the transfer of a protein to a membrane, and subsequent detection of the protein on the membrane. There are a variety of membranes suitable for use as western blot membranes known in the art including, without limitation, polyvinylidene difluoride (PVDF) membrane, nitrocellulose membrane, polyamide membrane, polyester membrane and nylon membrane. Western blotting typically utilizes a transfer buffering agent. There are a variety of western blot transfer buffering agents known in the art. In general, the western blot transfer buffering agent will have a pH that is above the isoelectric point of the protein to be transferred. Thus, when a voltage potential is applied, the protein will migrate toward the positive electrode. In one embodiment, the transfer buffering agent can have a pH below the isoelectric point of the protein to be transferred. In such cases, the protein will migrate toward the negative electrode.

The term “immunohistochemistry” refers to a technique for detecting the presence of an antigen with an antibody capable of specifically binding to the antigen in histological samples. The detection of the antibody-antigen complex is usually conducted by a chromogenic reaction with an enzyme-labeled antibody or by a fluorescent labeled antibody.

The term “telitacicept” is known under the trade name “Tai ai”, which has the amino acid sequence set forth in SEQ ID NO: 34:
(INN: https://extranet.who.int/soinn/mod/page/view.php?id=49)

| SEQ ID NO: 34 | |
|---|---|
| SRVDQEERFP QGLWTGVAMR SCPEEQYWDP LLGTCMSCKT ICNHQSQRTC AAFCRSLSCR | 60 |
| KEQGKFYDHL LRDCISCASI CGQHPKQCAY FCENKLRSPV NLPPELDKTH TCPPCPAPEA | 120 |
| EGAPSVFLFP PKPKDTLMIS RTPEVTCVVV DVSHEDPEVK FNWYVDGVEV HNAKTKPREE | 180 |
| QYNSTYRVVS VLTVLHQDWL NGKEYKCKVS NKALPSSIEK TISKAKGQPR EPQVYTLPPS | 240 |
| RDELTKNQVS LTCLVKGFYP SDIAVEWESN GQPENNYKTT PPVLDSDGSF FLYSKLTVDK | 300 |
| SRWQQGNVFS CSVMHEALHN HYTQKSLSLS PGK | 333 |

### EXAMPLES

The present disclosure will be further illustrated by way of the following examples. It should be noted that the following examples are for further elaboration and explanation of the present disclosure, and should not be regarded as limiting the present disclosure.

TACI antibodies were prepared by immunization with TACI antigen in the following examples.

### Example 1 TACI Antibody Preparation and Screening

After immunizing mice with human TACI antigen, spleen cells from the immunized mice were harvested and fused with mouse myeloma cells to establish a series of hybridoma cell lines.

The hybridoma cells were cultured, and the supernatants were used for enzyme-linked immunosorbent assay (ELISA). BLyS (R&D Systems) was coated on a 96-well microplate and incubated at 4° C. overnight. The plate was blocked with 300 μl of 3% BSA-PBST per well and incubated at 37° C. for 3 h. The supernatants were diluted at ratios of 10-fold, 100-fold and 1000-fold with PBST and separately incubated with 30 ng/ml telitacicept for a given period. These mixtures were then added to the 96-well plate at 100 μl per well and incubated at 37° C. for 1 h. Goat anti-human IgG Fc-HRP (1:5000 dilution) was added at 100 μl per well and incubated at 37° C. for 1 h. 100 μL of substrate per well was added for color development, and the absorbance was read at 450 nm and 655 nm. Antibodies with high activity were screened based on the difference in absorbance between 450 nm and 655 nm, respectively.

TABLE 1
Absorbance for supernatant from hybridoma cell line

| | Absorbance | | | |
|---|---|---|---|---|
| | Telitacicept + | Telitacicept + | Telitacicept + | |
| | 10-fold diluted | 100-fold diluted | 1000-fold diluted | |
| sample | supernatant | supernatant | supernatant | Telitacicept |
| 1B11D2-1 | 0.016 | 0.141 | 0.454 | 0.833 |
| 7G12F8-1 | 0.057 | 0.215 | 0.448 | 0.806 |
| 12C9F3-1 | 0.082 | 0.242 | 0.443 | 0.785 |
| 1B11C4-1 | 0.273 | 0.447 | 0.693 | 0.961 |
| 1E6F7-1 | 0.57 | 0.642 | 0.764 | 0.784 |
| 1G6G5-1 | 0.628 | 0.667 | 0.71 | 0.784 |
| 2G8G10-1 | 0.37 | 0.479 | 0.616 | 0.833 |
| 4C10C6-1 | 0.221 | 0.541 | 0.767 | 0.833 |
| 9H11H4-1 | 0.126 | 0.291 | 0.641 | 0.785 |
| 15A4C11-1 | 0.424 | 0.513 | 0.735 | 0.785 |

As shown in Table 1, the results showed that the culture supernatants of the three cell lines 1B11D2-1, 7G12F8-1 and 12C9F3-1 can significantly inhibit the binding of BlyS to the TACI-Fc fusion protein, and the other cell lines showed poor inhibition. The culture supernatants of the three cell lines were therefore collected and purified to obtain antibodies 1B11D2, 7G12F8 and 12C9F3.

Wherein, the amino acid sequences of antibodies 1B11D2, 7G12F8 and 12C9F3 are shown below.

Amino acid sequence of antibody 1B11D2:

| Heavy chain | SEQ ID NO: | EFSMH | |
|---|---|---|---|
| variable region | 1 | | |
| CDR1 | | | |
| (VH-CDR1) | | | |
| Heavy chain | SEQ ID NO: | GINPNHGGSSYNQKFKG | |
| variable region | 2 | | |
| CDR2 | | | |
| (VH-CDR2) | | | |
| Heavy chain | SEQ ID NO: | RGDYYGNLYYAMDY | |
| variable region | 3 | | |
| CDR3 | | | |
| (VH-CDR3) | | | |
| Light chain | SEQ ID NO: | KASQSVSNDVV | |
| variable region | 10 | | |
| CDR1 | | | |
| (VL-CDR1) | | | |
| Light chain | SEQ ID NO: | YASNRYT | |
| variable region | 11 | | |
| CDR2 | | | |
| (VL-CDR2) | | | |
| Light chain | SEQ ID NO: | QQDYSSPWA | |
| variable region | 12 | | |
| CDR3 | | | |
| (VL-CDR3) | | | |
| Heavy chain | SEQ ID NO: | MGWSWIFLFL LSGTAGVLSE VQLQQSGPEL VKPGASVKIS CKTSGYTFTE FSMHWVQQSH | 60 |
| variable region | 19 | GKSLEWIGGI NPNHGGSSYN QKFKGKATLT VDKSSSTAYM ELRSLTSEDS AVYYCARRGD | 120 |
| | | YYGNLYYAMDYWGQGTSVTVSS | 142 |
| Light chain | SEQ ID NO: | MKSQTQVFVF LLLCVSGAHG SIVMTQTPKF LLVSVGDSIT ITCKASQSVS NDVVWYQQKP | 60 |
| variable region | 22 | GQSPKLLIYY ASNRYTGVPD RFTGSGYGTD FTFTITTVQA EDLAVYFCQQ DYSSPWAFGG | 120 |
| | | GTKLEIKRA | 129 |
| Heavy chain | SEQ ID NO: | MGWSWIFLFL LSGTAGVLSE VQLQQSGPEL VKPGASVKIS CKTSGYTFTE FSMHWVQQSH | 60 |
| | 28 | GKSLEWIGGI NPNHGGSSYN QKFKGKATLT VDKSSSTAYM ELRSLTSEDS AVYYCARRGD | 120 |
| | | YYGNLYYAMD YWGQGTSVTV SSAKTTPPSV YPLAPGSAAQ TNSMVTLGCL VKGYFPEPVT | 180 |
| | | VTWNSGSLSS GVHTFPAVLQ SDLYTLSSSV TVPSSTWPSE TVTCNVAHPA SSTKVDKKIV | 240 |
| | | PRDCGCKPCI CTVPEVSSVF IFPPKPKDVL TITLTPKVTC VVVDISKDDP EVQFSWFVDD | 300 |
| | | VEVHTAQTQP REEQFNSTFR SVSELPIMHQ DWLNGKEFKC RVNSAAFPAP IEKTISKTKG | 360 |
| | | RPKAPQVYTI PPPKEQMAKD KVSLTCMITD FFPEDITVEW QWNGQPAENY KNTQPIMDTD | 420 |
| | | GSYFVYSKLN VQKSNWEAGN TFTCSVLHEG LHNHHTEKSL SHSPGK | 466 |
| Light chain | SEQ ID NO: | MKSQTQVFVF LLLCVSGAHG SIVMTQTPKF LLVSVGDSIT ITCKASQSVS NDVVWYQQKP | 60 |
| | 31 | GQSPKLLIYY ASNRYTGVPD RFTGSGYGTD FTFTITTVQA EDLAVYFCQQ DYSSPWAFGG | 120 |
| | | GTKLEIKRAD AAPTVSIFPP SSEQLTSGGA SVVCFLNNFY PKDINVKWKI DGSERQNGVL | 180 |
| | | NSWTDQDSKD STYSMSSTLT LTKDEYERHN SYTCEATHKT STSPIVKSFN RNEC | 1234 |

Amino acid sequence of antibody 7G12F8:

| Heavy chain | SEQ ID | AYALH | |
|---|---|---|---|
| variable region | NO: 4 | | |
| CDR1 | | | |
| (VH-CDR1) | | | |
| Heavy chain | SEQ ID | GINPNHGGVSYNQKFKD | |
| variable region | NO: 5 | | |
| CDR2 | | | |
| (VH-CDR2) | | | |
| Heavy chain | SEQ ID | FRWDLYYGMDY | |
| variable region | NO:6 | | |
| CDR3 | | | |
| (VH-CDR3) | | | |
| Light chain | SEQ ID | SDNSNMIYMY | |
| variable region | NO: 13 | | |
| CDR1 | | | |
| (VL-CDR1) | | | |
| Light chain | SEQ ID | DTSRLAS | |
| variable region | NO: 14 | | |
| CDR2 | | | |
| (VL-CDR2) | | | |
| Light chain | SEQ ID | HQRSIYSWT | |
| variable region | NO: 15 | | |
| CDR3 | | | |
| (VIL-CDR3) | | | |
| Heavy chain | SEQ ID | MGWSWIFLFL LSGTAGVLSE VQLQQSGPEL VRPGASVKKS CKTSGYTFTA YALHWVKQSH | 60 |
| variable region | NO: 20 | GTSLEWIGGI NPNHGGVSYN QKFKDKATLT VDKSSSTAYM ELRSLTSEDS AVYYCARFRW | 120 |
| | | DLYYGMDYWG QGTSVIVAS | 139 |
| Light chain | SEQ ID | MDFQVQIFSF LLISASVILS RGQIVLTQSP AIISVSSGEK VTMPCSDNSN MIYMYWNLWK | 60 |
| variable region | NO: 23 | PGTSPKRWIY DTSRLASGVP ARFSGSGFGT SFSLTVSSME AEDAATYYCH QRSIYSWTFG | 120 |
| | | GGTKLEIKRA | 130 |
| Heavy chain | SEQ ID | MGWSWIFLFL LSGTAGVLSE VQLQQSGPEL VRPGASVKKS CKTSGYTFTA YALHWVKQSH | 60 |
| | NO: 29 | GTSLEWIGGI NPNHGGVSYN QKFKDKATLT VDKSSSTAYM ELRSLTSEDS AVYYCARFRW | 120 |
| | | DLYYGMDYWG QGTSVIVASA KTTPPSVYPL APGSAAQTNS MVTLGCLVKG YFPEPVTVTW | 180 |
| | | NSGSLSSGVH TFPAVLQSDL YTLSSSVTVP SSTWPSETVT CNVAHPASST KVDKKIVPRD | 240 |
| | | CGCKPCICTV PEVSSVFIFP PKPKDVLTIT LTPKVTCVVV DISKDDPEVQ FSWFVDDVEV | 300 |
| | | HTAQTQPREE QFNSTFRSVS ELPIMHQDWL NGKEFKCRVN SAAFPAPIEK TISKTKGRPK | 360 |
| | | APQVYTIPPP KEQMAKDKVS LTCMITDFFP EDITVEWQWN GQPAENYKNT QPIMDTDGSY | 420 |
| | | FVYSKLNVQK SNWEAGNTFT CSVLHEGLHN HHTEKSLSHS PGK | 463 |
| Light chain | SEQ ID | MDFQVQIFSF LLISASVILS RGQIVLTQSP AIISVSSGEK VTMPCSDNSN MIYMYWNLWK | 60 |
| | NO: 32 | PGTSPKRWIY DTSRLASGVP ARFSGSGFGT SFSLTVSSME AEDAATYYCH QRSIYSWTFG | 120 |
| | | GGTKLEIKRA DAAPTVSIFP PSSEQLTSGG ASVVCFLNNF YPKDINVKWK IDGSERQNGV | 180 |
| | | LNSWTDQDSK DSTYSMSSTL TLTKDEYERH NSYTCEATHK TSTSPIVKSF NRNEC | 235 |

Amino acid sequence of antibody 12C9F3:

| Heavy chain | SEQ ID | EYAMH | |
|---|---|---|---|
| variable region | NO: 7 | | |
| CDR1 | | | |
| (VH-CDR1) | | | |
| Heavy chain | SEQ ID | GINPNHGGTSNNQKFKG | |
| variable region | NO: 8 | | |
| CDR2 | | | |
| (VH-CDR2) | | | |
| Heavy chain | SEQ ID | WGTDPFFAY | |
| variable region | NO: 9 | | |
| CDR3 | | | |
| (VH-CDR3) | | | |
| Light chain | SEQ ID | KASQDVSTAVA | |
| variable region | NO: 16 | | |
| CDR1 | | | |
| (VL-CDR1) | | | |
| Light chain | SEQ ID | WASTRHT | |
| variable region | NO: 17 | | |
| CDR2 | | | |
| (VL-CDR2) | | | |
| Light chain | SEQ ID | HQHYAPPWT | |
| variable region | NO: 18 | | |
| CDR3 | | | |
| (VL-CDR3) | | | |
| Heavy chain | SEQ ID | MGWSWIFLFL LSGTAGVLSE VQLQQSGPEL VKPGASVKIS CKTSGYTFTE YAMHWVKQSH | 60 |
| variable region | NO: 21 | GKSLEWIGGI NPNHGGTSNN QKFKGKATLT VDKSSSTAYM DFRSLTSEDS AVYYCARWGT | 120 |
| | | DPFFAYWGQG TLVTVSA | 137 |
| Light chain | SEQ ID | MESQIQAFVF VFLWLSGVDG DIVMTQSHKF MSTSVGDRVS ITCKASQDVS TAVAWYQQKP | 60 |
| variable region | NO: 24 | GQSPKLLIYW ASTRHTGVPD RFTGSGSGTD YTLTITSVQA EDLTLYYCHQ HYAPPWTFGG | 120 |
| | | GTKLEIKRA | 129 |
| Heavy chain | SEQ ID | MGWSWIFLFL LSGTAGVLSE VQLQQSGPEL VKPGASVKIS CKTSGYTFTE YAMHWVKQSH | 60 |
| | NO: 30 | GKSLEWIGGI NPNHGGTSNN QKFKGKATLT VDKSSSTAYM DFRSLTSEDS AVYYCARWGT | 120 |
| | | DPFFAYWQG TLVTVSAAKT TPPSVYPLAP GSAAQTNSMV TLGCLVKGYF PEPVTVTWNS | 180 |
| | | GSLSSGVHTF PAVLQSDLYT LSSSVTVPSS TWPSETVTCN VAHPASSTKV DKKIVPRDCG | 240 |
| | | CKPCICTVPE VSSVFIFPPK PKDVLTITLT PKVTCVVVDI SKDDPEVQFS WFVDDVEVHT | 300 |
| | | AQTQPREEQF NSTFRSVSEL PIMHQDWLNG KEFKCRVNSA AFPAPIEKTI SKTKGRPKAP | 360 |
| | | QVYTIPPPKE QMAKDKVSLT CMITDFFPED ITVEWQWNGQ PAENYKNTQP IMDTDGSYFV | 420 |
| | | YSKLNVQKSN WEAGNTFTCS VLHEGLHNHH TEKSLSHSPG K | 461 |
| Light chain | SEQ ID | MESQIQAFVF VFLWLSGVDG DIVMTQSHKF MSTSVGDRVS ITCKASQDVS TAVAWYQQKP | 60 |
| | NO: 33 | GQSPKLLIYW ASTRHTGVPD RFTGSGSGTD YTLTITSVQA EDLTLYYCHQ HYAPPWTFGG | 120 |
| | | GTKLEIKRAD AAPTVSIFPP SSEQLTSGGA SVVCFLNNFY PKDINVKWKI DGSERQNGVL | 180 |
| | | NSWTDQDSKD STYSMSSTLT LTKDEYERHN SYTCEATHKT STSPIVKSFN RNEC | 234 |

### Example 2: Analysis of Binding Epitope

### I. Preparation of Test Substance

1. Buffer composition: 20 mM PB, 75 mM NaCl (PH 7.4), H₂O

2. Test substance A was obtained by diluting 1B11D2 with buffer to 40 μM.

3. Test substance B was obtained by diluting BLyS (R&D Systems) with buffer to 40 μM.

4. Test substance C was obtained by diluting telitacicept with buffer to 40 μM.

5. Interaction group A was obtained by mixing test substance A and an equal volume of test substance C well.

6. Interaction group B was obtained by mixing test substance B and an equal volume of test substance C well.

7. Control group A-1 was obtained by mixing test substance C and an equal volume of buffer well.

8. Control group B-1 was obtained by mixing test substance C and an equal volume of buffer well.

### II. Liquid Spectrometry and Mass Spectrometry Detection

Interacting group A, interacting group B, control group A-1 and control group B-1 were digested with pepsin. The UPLC (Ultra-performance liquid chromatography) system (Ultimate NCS-3500 RSLC pump system, ThermoFisher) was used for separation (a mobile phase of 0.1% FA/H₂O aqueous solution as liquid A and 0.1% FA/90% ACN/10% H₂O solution as liquid B, see Table 2 for the gradient used in liquid chromatography).

TABLE 2
Gradient setting in liquid chromatography

| | | flow rate | |
|---|---|---|---|
| number | time (min) | (μl/min) | % liquid B |
| 1 | 0.0 | 45 | 2.0 |
| 2 | 1.0 | 45 | 2.0 |
| 3 | 4.0 | 45 | 2.0 |
| 4 | 4.1 | 45 | 10.0 |
| 5 | 14.0 | 45 | 40.0 |
| 6 | 14.1 | 45 | 90.0 |
| 7 | 16.0 | 45 | 90.0 |
| 8 | 16.1 | 45 | 2.0 |
| 9 | 18.0 | 45 | 2.0 |

The digested products were loaded using an automatic sampler (PAL3 autosampler, LEAP Technologies), separated through a chromatography column (ACQUITY UPLC Peptide CSH C18 Column, 130 Å, 1.7 μm, 1 mm×50 mm, Thermo Fisher), scanned and analyzed by mass spectrometry using a high-resolution mass spectrometer (Orbitrap Fusion, Thermo Fisher). Mass spectrometry analysis was detailed below: scan time: 15 min; detection mode: positive ion and parent ion scan range: 300-1500 m/z. Resolutions for first-stage mass spectrometry and second-stage mass spectrometry were 60,000 and 15,000, respectively. Parameters for reaction and detection are shown in Table 3.

TABLE 3
Reaction parameter

| Reaction temperature | 15° C. | reaction | 20 mM PB, 75 mM NaCl, PH 7.4 (H₂O or D₂O) | | |
|---|---|---|---|---|---|
| | | buffer | | | |
| Reaction time | 0 s, 10 s, 30 s, 200 s, 600 s, 1200 s and 3600 s (three | | | | |
| | technical replicates were set for each time point) | | | | |
| Quenching temperature | 0.5° C. | Quenching | 4M Guanidine Hydrochloride, 0.25M | | |
| | | buffer | TCEP, 100 mM Citric Acid, PH 2.3 | | |
| Digestion temperature | 4° C. | Digestion | 180 s | Digestion | (On-line digestion, |
| | | time | | buffer | liquid A) |

| | |
|---|---|
| Separation temperature | 0.5° C. |
| in liquid chromatography | |

### III. Detection Results

### Detection Results of Interaction Group B

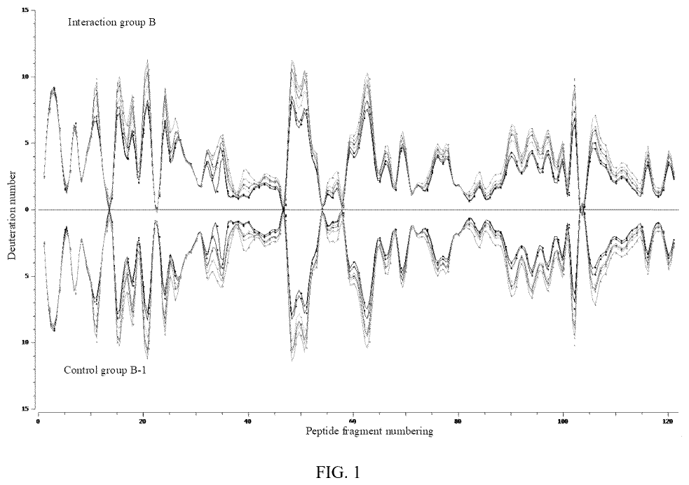

FIG. 1 is a butterfly plot analyzing the deuteration consistency between control group B-1 and interaction group B. According to the detection results, the butterfly plot had a good overall symmetry with local differences.

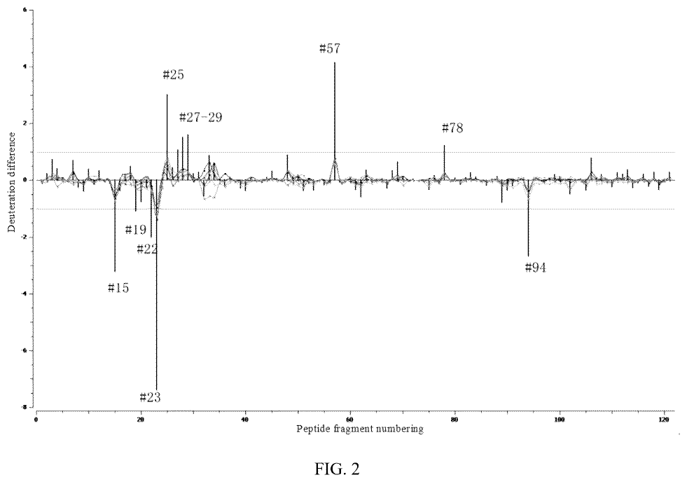

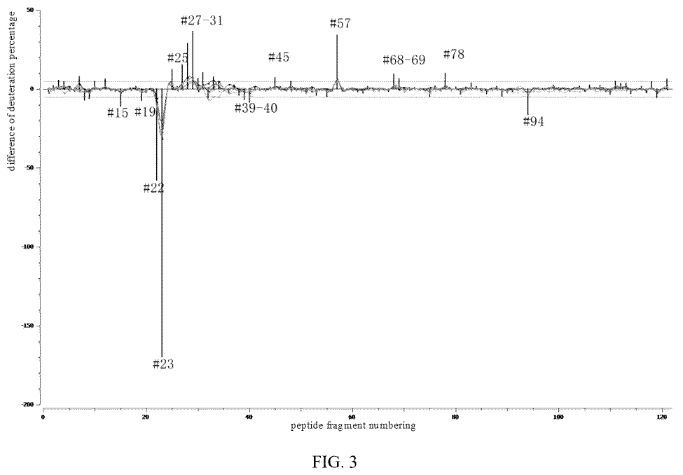

FIG. 2 and FIG. 3 are residue plots using different ordinates of deuteration level (in Da) and deuteration difference percentage versus peptide numbering. FIG. 2 shows a significant difference in the deuteration between the control group B-1 and the interaction group B (Horizontal line represents a threshold of 1 Da). Peptide regions showing a significant difference were the following regions: 29-64 (#15), 50-68 (#19), 67-72 (#22), 67-73 (#23), 68-99 (#25), 94-106 (#27), 96-106 (#28), 98-106 (#29), 193-208 (#57), 235-252 (#78) and 267-290 (#94), indicating significant structural changes. FIG. 3 shows a significant difference in the deuteration between the control group B-1 and the interaction group B (Horizontal line represents a 5% threshold). Peptide regions showing a significant difference were the following regions: 29-64 (#15), 50-68 (#19), 67-72 (#22), 67-73 (#23), 68-99 (#25), 94-106 (#27), 96-106 (#28), 98-106 (#29), 99-106 (#30), 100-106 (#31), 128-137 (#39), 132-138 (#40), 156-162 (#45), 193-208 (#57), 234-241 (#68), 234-248 (#69), 235-252 (#78) and 267-290 (#94), indicating significant structural changes.

Therefore, in the case that the butterfly plot of control group B-1 and interaction group B had good overall symmetry and local differences, a comprehensive analysis of the residue plots in different statistical approaches and deuteration difference of overall peptide in FIG. 2 and FIG. 3 concluded that antigen-antibody interactions may occur in regions 29-32, 67-73 and 267-290 and their vicinity, wherein region 67-73 showed the highest significant difference in reduced deuteration level.

### Detection Results of Interaction Group A

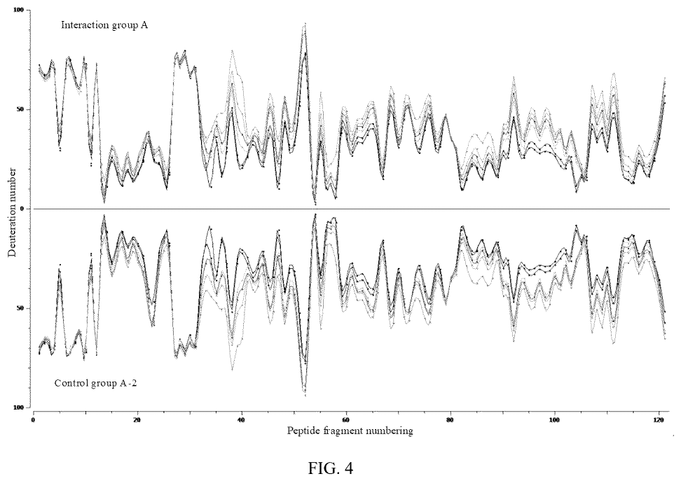

FIG. 4 is a butterfly plot analyzing the deuteration consistency between control group A-1 and interaction group A. According to the detection results, the butterfly plot of control group A-1 and interaction group A had a good overall symmetry with local differences.

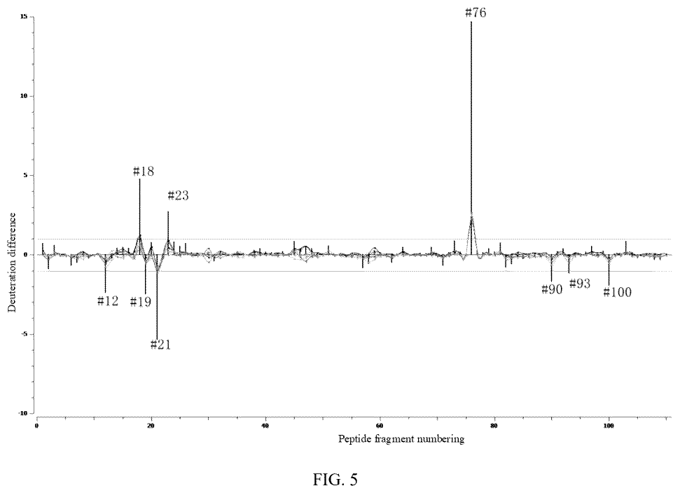

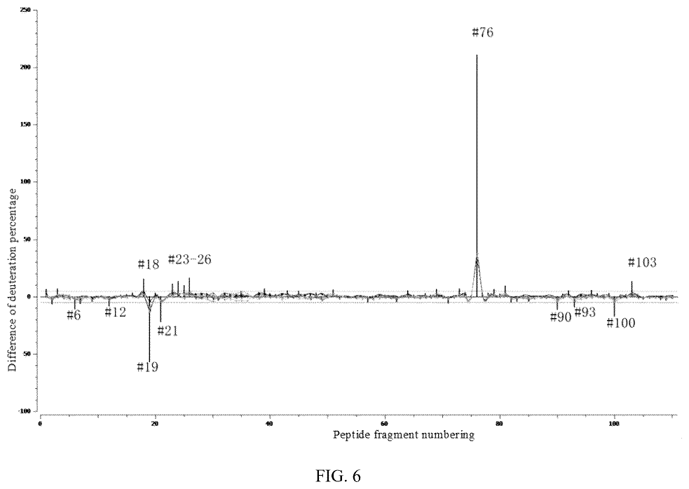

FIG. 5 and FIG. 6 are residue plots using different ordinates of deuteration level (in Da) and deuteration difference percentage versus peptide numbering. FIG. 5 shows a significant difference in the deuteration between the control group A-1 and the interaction group A. Peptide regions showing a significant difference were the following regions: 29-64 (#12), 54-92 (#18), 67-73 (#19), 68-99 (#21), 74-107 (#23), 252-262 (#76), 269-290 (#90), 271-291 (#93) and 297-311 (#100), indicating significant structural changes. FIG. 6 shows a significant difference in the deuteration between the control group A-1 and the interaction group A. Peptide regions showing a significant difference were the following regions: 7-16 (#6), 29-64 (#12), 54-92 (#18), 67-73 (#19), 68-99 (#21), 74-107 (#23), 95-106 (#24), 252-262 (#76), 269-290 (#90), 297-311 (#93) and 301-309 (#103), indicating significant structural changes.

Therefore, in the case that the butterfly plot of control group A-1 and interaction group A had good overall symmetry and local differences, a comprehensive analysis of the residue plots with different types of threshold and deuteration difference of overall peptide in FIG. 5 and FIG. 6 concluded that antigen-antibody interactions may occur in regions 29-32, 67-73 and 269-290 and their vicinity, wherein region 67-73 showed the highest significant difference in reduced deuteration level.

### IV. Conclusion

According to the butterfly plot analyzing the deuteration consistency between control B-1 and interaction group B and their residual plots showing the deuteration level difference and the deuteration difference percentage, it can be seen that the interaction occurred in regions 29-32, 67-73 and 267-290 and their vicinity, which were the ligand-receptor binding epitopes for interaction group B, with region 67-73 showing the highest affinity for the interaction group.

According to the butterfly plot analyzing the deuteration consistency between control A-1 and interaction group A and their residual plots showing the deuteration level difference and the deuteration difference percentage, it can be seen that the interaction occurred in regions 29-32, 67-73 and 269-290 and their vicinity, which were the ligand-receptor binding epitopes for interaction group A, with region 67-73 showing the highest affinity for the interaction group.

Interaction group B and interaction group A used the same regions 29-32, 67-73 and 269-290 as binding epitopes, indicating a competitive binding between 1B11D2 and BLyS to telitacicept and a high degree of epitope overlap.

### Example 3: Affinity for TACI

An Octet RH16 molecular interaction analyzer (Sartorius) was used. An appropriate amount of antibody 1B11D2 reacted with EZ-Link Sulfo-NHS-LC-LC-Biotin (Sartorius, 18-5019) at room temperature for 30 min followed by centrifugation with a Zeba spin desalting column to collect the biotinylated antibody 1B11D2.

The biotinylated antibody 1B11D2 was immobilized on Octet SA Biosensors (Thermo, 87766) and telitacicept at various concentrations was used as the analyte. The detection curve was fitted by kinetic analysis to obtain the results.

TABLE 4
Result of affinity measurement of antibody 1B11D2 to telitacicept

| K_{D} | kₐ (1/Ms) | Kdis (1/s) |
|---|---|---|
| 3.246E−10 | 3.229E+5 | 1.048E−4 |

Table 4 shows the results of affinity measurement of the antibody 1B11D2 to telitacicept. The experimental results showed that antibody 1B11D2 can specifically bind to telitacicept with a strong binding affinity between the two. Antibodies 7G12F8 and 12C9F3 also showed good binding affinity for telitacicept as measured by this method.

### Example 4 Measurement of Free TACI Content

### I. Sample Preparation

Standards: Telitacicept was prepared to the following concentrations: 1000.0 ng/ml, 500.0 ng/ml, 400.0 ng/ml, 300.0 ng/ml, 200.0 ng/ml, 120.0 ng/ml, 60.0 ng/ml, 30.0 ng/ml, 15.0 ng/ml and 10.0 ng/ml.

Sample to be tested: 15 μl of clinical blood sample was taken and mixed well with 105 μl of 1% BSA-PBS to obtain a mixture, and 10 μl of the mixture was mixed well with 90 μl of 1% BSA-PBS.

Blank control: 15 μl of blank serum was taken and mixed well with 105 μl of 1% BSA-PBS to obtain a mixture, and 10 μl of the mixture was mixed well with 90 μl of 1% BSA-PBS.

### II. Measurement of Free TACI Content

A. Coating: Antibody 1B11D2 (14.2 μg/ml) was coated onto a 96-well microplate and incubated at 4° C. overnight.

B. Blocking: The plate was washed, blocked with 300 μl of 3% BSA-PBST per well and incubated at 37° C. for 2h.

C. Detection of antibody binding: The plate was washed. Standards, the sample to be tested and blank control were added at 100 μl per well and incubated at room temperature for 1.5 h to allow complete binding of free TACI in each sample to the antibody 1B11D2.

D. Binding of secondary antibody: The plate was washed. The secondary antibody goat anti-human IgG Fc (Biotin) was diluted at 1:5000, added to the microplate at 100 μl per well and incubated at room temperature for 40 min.

E. Color development of secondary antibody: The plate was washed. Streptavidin-HRP was added and incubated at room temperature for 40 min.

F. Color development of substrate: The plate was washed. 100 μl of substrate (tetramethylbenzidine) per well was added for color development.

G. Terminating: 50 μl of stop solution (2M H₂SO₄) per well was added to terminate the reaction.

H. Reading: The difference of values was read at 450 nm and 570 nm by using a microplate reader.

### III. Plotting of Standard Curve

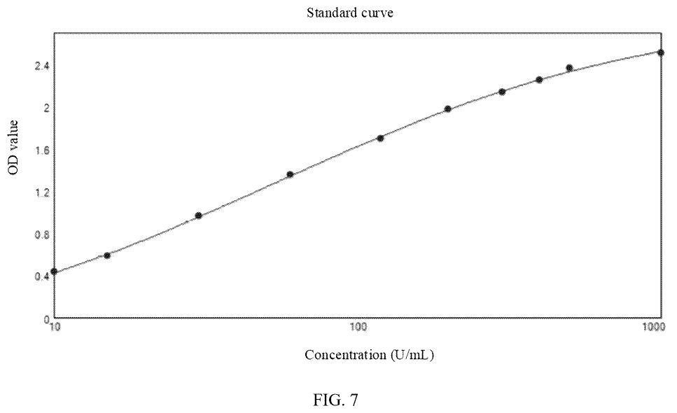

See Table 5 for the OD values of the standards. The standard curve was plotted with the concentrations of the standards as the abscissa and the OD value as the ordinate, as shown in FIG. 7.

TABLE 5
Standard curve for in vitro detection of antibody 1B11D2

| Concentration of free- | |
|---|---|
| telitacicept in sample (ng/ml) | OD (Mean Value) |
| 10.0 | 0.429 |
| 15.0 | 0.584 |
| 30.0 | 0.967 |
| 60.0 | 1.352 |
| 120.0 | 1.700 |
| 200.0 | 1.973 |
| 300.0 | 2.134 |
| 400.0 | 2.250 |
| 500.0 | 2.361 |
| 1000.0 | 2.509 |

### IV. Calculation of Free TACI Content in the Sample to be Tested

The OD value of the sample to be tested was 1.977 as read by using the microplate reader, and the content of free TACI in the sample to be tested was 207.275 (ng/ml) as calculated according to the formula below.

$$y = D + \frac{A - D}{1 + {( \frac{X}{C} )}^{B}}$$

Wherein y represents the OD value, x represents a concentration, A represents asymptotic upper bound estimate, B represents the slope, C represents the concentration corresponding to half-maximal binding (EC₅₀), and D represents asymptotic lower bound estimate. In this study, the TACI antibody can be used for detecting the content of TACI in patients with immune diseases, particularly the content of free TACI or TACI-Fc fusion protein drugs. The effective evaluation of drug absorption according to the drug content in patients played a role in assessing the condition of patients with autoimmune diseases, monitoring therapeutic efficacy and assessing prognosis.

The above description is only for some embodiments by way of example only and without limitation to the combination of features necessary for carrying the present disclosure into effect. The headings provided herein are not intended to limit the various embodiments of the present disclosure. Terms such as “including”, “comprising” and “containing” are not intended to be limiting. In addition, unless otherwise indicated, the singular form “a”, “an”, or “the” includes plural references, as well as “or” means “and/or”. Unless otherwise defined herein, all technical and scientific terms used herein have the same meaning as commonly understood in the art.

All publications and patents mentioned in the present application are incorporated herein by reference. Without departing from the scope and spirit of the present disclosure, various modifications and variations of the described method and composition of the present disclosure will be apparent in the art. Although the present disclosure has been described by using some embodiments, it should be understood that the claimed disclosure should not be unduly limited to these embodiments. In fact, many variations of the described modes for carrying out the embodiments are intended to be included within the scope of the appended claims.

1. An antibody or antigen-binding fragment thereof, capable of binding specifically to TACI, wherein the antibody or antigen-binding fragment thereof comprises:
(1) a heavy chain variable region (VH) comprising VH-CDR1 having an amino acid sequence of SEQ ID NO: 1, VH-CDR2 having an amino acid sequence of SEQ ID NO: 2 and VH-CDR3 having an amino acid sequence of SEQ ID NO: 3, or
(2) a heavy chain variable region (VH) comprising VH-CDR1 having an amino acid sequence of SEQ ID NO: 4, VH-CDR2 having an amino acid sequence of SEQ ID NO: 5 and VH-CDR3 having an amino acid sequence of SEQ ID NO: 6, or
(3) a heavy chain variable region (VH) comprising VH-CDR1 having an amino acid sequence of SEQ ID NO: 7, VH-CDR2 having an amino acid sequence of SEQ ID NO: 8 and VH-CDR3 having an amino acid sequence of SEQ ID NO: 9; and/or
(4) a light chain variable region (VL) comprising VL-CDR1 having an amino acid sequence of SEQ ID NO: 10, VL-CDR2 having an amino acid sequence of SEQ ID NO: 11 and VL-CDR3 having an amino acid sequence of SEQ ID NO: 12,
(5) a light chain variable region (VL) comprising VL-CDR1 having an amino acid sequence of SEQ ID NO: 13, VL-CDR2 having an amino acid sequence of SEQ ID NO: 14 and VL-CDR3 having an amino acid sequence of SEQ ID NO: 15, or
(6) a light chain variable region (VL) comprising VL-CDR1 having an amino acid sequence of SEQ ID NO: 16, VL-CDR2 having an amino acid sequence of SEQ ID NO: 17 and VL-CDR3 having an amino acid sequence of SEQ ID NO: 18.

2. The antibody or antigen-binding fragment thereof according to claim 1, wherein the antibody is a mouse antibody, a rabbit antibody, a murine-derived antibody or a rabbit-derived antibody, or
the antigen-binding fragment is Fab, F(ab′)₂, Fab′, Fv or ScFv.

3. (canceled)

4. The antibody or antigen binding fragment thereof according to claim 1, wherein the antibody or antigen binding fragment thereof comprises:
(1) a heavy chain variable region (VH), wherein the heavy chain variable region has at least 95%, 96%, 97%, 98%, 99% or 100% sequence identity to the amino acid sequence set forth in SEQ ID NO: 19, SEQ ID NO: 20 or SEQ ID NO: 21, and/or
(2) a light chain variable region (VL), wherein the light chain variable region has at least 95%, 96%, 97%, 98%, 99% or 100% sequence identity to the amino acid sequence set forth in SEQ ID NO: 22, SEQ ID NO: 23 or SEQ ID NO: 24.

5. The antibody or antigen binding fragment thereof according to claim 4, wherein the antibody or antigen binding fragment thereof comprises:
(1) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 19, and the light chain variable region has at least 95%, 96%, 97%, 98%, 99% or 100% sequence identity to the amino acid sequence set forth in SEQ ID NO: 22,
(2) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 20, and the light chain variable region has at least 95%, 96%, 97%, 98%, 99% or 100% sequence identity to the amino acid sequence set forth in SEQ ID NO: 23, or
(3) a heavy chain variable region (VH) and/or a light chain variable region (VL), wherein the heavy chain variable region has at least 95%, 96%, 97%, 98%, 99% or 100% sequence identity to the amino acid sequence set forth in SEQ ID NO: 21, and the light chain variable region has at least 95%, 96%, 97%, 98%, 99% or 100% sequence identity to the amino acid sequence set forth in SEQ ID NO: 24.

6. The antibody or antigen binding fragment thereof according to claim 1, wherein the antibody or antigen binding fragment thereof comprises an Fc region, preferably, the Fc region comprises an amino acid sequence having at least 95%, 96%, 97%, 98%, 99%, or 100% sequence identity to the amino acid sequence set forth in SEQ ID NO: 25, SEQ ID NO: 26 or SEQ ID NO: 27.

7. (canceled)

8. The antibody or antigen binding fragment thereof according to claim 1, wherein the antibody or antigen binding fragment thereof comprises:
(1) a heavy chain, wherein the heavy chain has at least 95%, 96%, 97%, 98%, 99%, or 100% sequence identity to the amino acid sequence set forth in SEQ ID NO: 28, SEQ ID NO: 29 or SEQ ID NO: 30; and/or
(2) a light chain, wherein the light chain has at least 95%, 96%, 97%, 98%, 99%, or 100% sequence identity to the amino acid sequence set forth in SEQ ID NO: 31, SEQ ID NO: 32 or SEQ ID NO: 33.

9. The antibody or antigen binding fragment thereof according to claim 8, wherein the antibody or antigen binding fragment thereof comprises:
(1) a heavy chain and/or a light chain, wherein the heavy chain has at least 95%, 96%, 97%, 98%, 99% or 100% sequence identity to the amino acid sequence set forth in SEQ ID NO: 28, and the light chain has at least 95%, 96%, 97%, 98%, 99% or 100% sequence identity to the amino acid sequence set forth in SEQ ID NO: 31; or
(2) a heavy chain and/or a light chain, wherein the heavy chain has at least 95%, 96%, 97%, 98%, 99% or 100% sequence identity to the amino acid sequence set forth in SEQ ID NO: 29, and the light chain has at least 95%, 96%, 97%, 98%, 99% or 100% sequence identity to the amino acid sequence set forth in SEQ ID NO: 32; or
(3) a heavy chain and/or a light chain, wherein the heavy chain has at least 95%, 96%, 97%, 98%, 99% or 100% sequence identity to the amino acid sequence set forth in SEQ ID NO: 30, and the light chain has at least 95%, 96%, 97%, 98%, 99% or 100% sequence identity to the amino acid sequence set forth in SEQ ID NO: 33.

10. The antibody or antigen binding fragment thereof according to claim 1, wherein the antibody or antigen binding fragment thereof can be further conjugated with a detectable label.

11. The antibody or antigen binding fragment thereof according to claim 10, wherein the detectable label is an enzyme, or
the detectable label is a light-emitting label, or
the detectable label is a radioisotope, or.
the detectable label is a chromogenic label, or
the detectable label is a hapten, or
the detectable label is a metal complex.

12-16. (canceled)

17. A method of measuring TACI content in a sample, comprising using the antibody or antigen-binding fragment thereof according to claim 1, preferably, the sample is a biological sample, more preferably, the sample is a blood sample.

18-19. (canceled)

20. The method according to claim 17, wherein the sample is derived from a subject overexpressing BLyS and APRIL, preferably, the sample is derived from a subject with an autoimmune disease, more preferably, the autoimmune disease is one or more selected from the group consisting of rheumatoid arthritis, rheumatic arthritis, juvenile rheumatoid arthritis, systemic lupus erythematosus (SLE), lupus nephritis (LN), Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathy, myasthenia gravis, vasculitis, diabetes mellitus, Raynaud's syndrome, Sjogren's syndrome, glomerular nephritis, autoimmune hepatitis, autoimmune thyroiditis, neuromyelitis optica and sicca syndrome.

21-22. (canceled)

23. The method according to claim 20, wherein the subject is a human.

24. The method according to claim 17, wherein the measurement is carried out by ELISA, or
the measurement is carried out by Western blot, or
the measurement is carried out by immunohistochemistry (IHC).

25-26. (canceled)

27. A method for measuring TACI content in a sample comprising steps of contacting a sample with the antibody or antigen-binding fragment thereof according to claim 1, and detecting the existence of a bound antibody or antigen-binding fragment thereof.

28. An isolated polynucleotide encoding the antibody or antigen-binding fragment thereof according to claim 1.

29. An expression vector comprising the polynucleotide according to claim 28.

30. (canceled)

31. A host cell comprising at least one expression vector according to claim 29.

32. A method of manufacturing a kit, comprising using the antibody or antigen-binding fragment thereof according to claim 1.

33. A kit comprising the antibody according to claim 1.
